(19) United States
(12) Patent Application Publication
(10) Pub. No.: US 2020/0370069 A1
(43) Pub. Date: Nov. 26, 2020

(54) TREATMENT OF SPINAL MUSCULAR ATROPHY

(71) Applicants: GENETHON, EVRY (FR); INSERM (INSTITUT NATIONAL DE LA SANTÉ ET DE LA RECHERCHE MÉDICALE), PARIS (FR); UNIVERSITE D'EVRY VAL D'ESSONNE, EVRY (FR)

(72) Inventors: ANA BUJ BELLO, PARIS (FR); MARTINA MARINELLO, CHOISY LE ROI (FR); SAMIA MARTIN, SAVIGNY SUR ORGE (FR)

(21) Appl. No.: 16/629,339

(22) PCT Filed: Jul. 6, 2018
(86) PCT No.: PCT/EP2018/068434

§ 371 Date: Jan. 8, 2020

(30) Foreign Application Priority Data
Jul. 8, 2017 (EP) 17305895.9

(51) Int. Cl. C12N 15/86; A61P 25/00; C07K 14/47; A61K 38/17; A61K 48/00

(52) U.S. Cl. CPC C12N 15/86; A61P 25/00; C07K 14/47; C12N 2750/14171; A61K 48/0066; C12N 2750/14143; A61K 38/1709

(57) ABSTRACT

The present invention relates to a recombinant adeno-associated virus (rAAV) vector comprising a serotype 9 or rh10 AAV capsid, for use in a method for the treatment of spinal muscular atrophy (SMA).

### FIELD OF THE INVENTION

The present invention relates to a recombinant adeno-associated virus (rAAV) vector comprising a serotype 9 or rh10 AAV capsid, for use in a method for the treatment of spinal muscular atrophy (SMA).

### BACKGROUND OF THE INVENTION

Spinal Muscular Atrophy (“SMA”), in its broadest sense, describes a collection of inherited and acquired central nervous system (CNS) diseases characterized by motor neuron loss in the spinal cord causing muscle weakness and atrophy. The most common form of SMA is caused by mutation of the Survival Motor Neuron (“SMN”) gene, and manifests over a wide range of severity affecting infants through adults. Infantile SMA is one of the most severe forms of this neurodegenerative disorder. The onset is usually sudden and dramatic. Some of the symptoms include: muscle weakness, poor muscle tone, weak cry, limpness or a tendency to flop, difficulty sucking or swallowing, accumulation of secretions in the lungs or throat, feeding difficulties and increased susceptibility to respiratory tract infections. The legs tend to be weaker than the arms and developmental milestones, such as lifting the head or sitting up, cannot be reached. In general, the earlier the symptoms appear, the shorter the lifespan. Shortly after symptoms appear, the motor neuron cells quickly deteriorate. The disease can be fatal. The course of SMA is directly related to the severity of weakness. Infants with a severe form of SMA frequently succumb to respiratory disease due to weakness in the muscles that support breathing. Children with milder forms of SMA live much longer, although they may need extensive medical support, especially those at the more severe end of the spectrum. Disease progression and life expectancy strongly correlate with the subject's age at onset and the level of weakness. The clinical spectrum of SMA disorders has been divided into the following five groups:

(a) Neonatal SMA (Type 0 SMA; before birth): Type 0, also known as very severe SMA, is the most severe form of SMA and begins before birth. Usually, the first symptom of type 0 is reduced movement of the fetus that is first seen between 30 and 36 weeks of the pregnancy. After birth, these newborns have little movement and have difficulties with swallowing and breathing.

(b) Infantile SMA (Type 1 SMA or Werdnig-Hoffmann disease; generally 0-6 months): Type 1 SMA, also known as severe infantile SMA or Werdnig Hoffmann disease, is very severe, and manifests at birth or within 6 months of life. Patients never achieve the ability to sit, and death usually occurs within the first 2 years without ventilatory support.

(c) Intermediate SMA (Type 2 SMA or Dubowitz disease; generally 6-18 months): Patients with Type 2 SMA, or intermediate SMA, achieve the ability to sit unsupported, but never stand or walk unaided. The onset of weakness is usually recognized sometime between 6 and 18 months. Prognosis in this group is largely dependent on the degree of respiratory involvement.

(d) Juvenile SMA (Type 3 or Kugelberg-Welander disease; generally >18 months): Type 3 SMA describes those who are able to walk independently at some point during their disease course, but often become wheelchair bound during youth or adulthood.

(e) Adult SMA (Type 4 SMA): Weakness usually begins in late adolescence in tongue, hands, or feet then progresses to other areas of the body. The course of adult disease is much slower and has little or no impact on life expectancy.

The SMA disease gene has been mapped by linkage analysis to a complex region of chromosome 5q. In humans, this region has a large inverted duplication; consequently, there are two copies of the SMN gene. SMA is caused by a recessive mutation or deletion of the telomeric copy of the gene SMN1 in both chromosomes, resulting in the loss of SMN1 gene function. However, most patients retain a centromeric copy of the gene SMN2, and its copy number in SMA patients has been implicated as having an important modifying effect on disease severity; i.e., an increased copy number of SMN2 is observed in less severe disease. Nevertheless, SMN2 is unable to compensate completely for the loss of SMN1 function, because the SMN2 gene produces reduced amounts of full-length RNA and is less efficient at making protein, although, it does so in low amounts. More particularly, the SMN1 and SMN2 genes differ by five nucleotides; one of these differences—a translationally silent C to T substitution in an exonic splicing region—results in frequent exon 7 skipping during transcription of SMN2. As a result, the majority of transcripts produced from SMN2 lack exon 7 (SMNΔEx7), and encode a truncated protein which is rapidly degraded (about 10% of the SMN2 transcripts are full length and encode a functional SMN protein).

As a consequence, gene replacement of SMN1 was proposed as a strategy for the treatment of SMA. In particular, focus was previously made on the treatment of SMA by delivery of the SMN gene across the blood-brain barrier with a double-stranded self-complementary AAV9 vector administered via the systemic route (such as in WO2010/071832). Indeed, AAV vectors comprising an AAV9 capsid were shown to be capable of crossing the blood-brain barrier and to then transduce cells involved in SMA development such as motor neurons and glial cells. In application WO2014/022582, it was proposed to intrathecally inject a recombinant double-stranded self-complementary AAV9 vector to a subject in need thereof, along with a non-ionic, low-osmolar contrast agent to improve transduction efficiency. However, it can be seen from the prior art that the general teaching was that AAV9 vectors comprising a double-stranded, self-complementary genome were the optimal vectors for providing the best transduction in the central nervous system (CNS). Self-complementary vectors do not require second-strand synthesis, which is a rate limiting step of AAV vectors. Since the description by McCarty et al. (McCarty et al., Gene Ther., 2001, 8(16):1248-54; McCarthy et al., Gene Ther., 2003, 10(26): 2112-2118) of a 5- to 140-fold more efficient transduction with self-complementary AAV vectors, these vectors were selected as vectors of choice for achieving massive and rapid expression of a transgene delivered with a recombinant AAV vector. Self-complementary rAAV vectors are currently considered as much more efficient than single-stranded vectors at lower doses. Accordingly, those skilled in the art were incited to implement rAAV vectors comprising a double-stranded, self-complementary genome rather than an rAAV vector comprising a single-stranded genome, considering that better expression of a SMN protein was anticipated to be obtained with the former, and thus better treatment.

Against this prejudice, it is herein shown that an AAV vector comprising an AAV9 or AAVrh10 capsid and a single-stranded genome is able to considerably increase survival of a mouse model of SMA.

### SUMMARY OF THE INVENTION

The present invention relates to a recombinant adeno-associated virus (rAAV) vector comprising
(i) an AAV9 capsid or an AAVrh10 capsid; and
(ii) a single-stranded genome including a gene coding a spinal motor neuron (SMN) protein,
for use in a method for the treatment of spinal muscular atrophy (SMA).

In a particular embodiment, said SMN protein is derived from the human SMN1 gene.

In a further particular embodiment, said rAAV vector comprises an AAV9 capsid.

In another embodiment, said rAAV vector is administered into the cerebrospinal fluid of a subject, in particular by intrathecal and/or intracerebroventricular injection.

In another embodiment, said SMA is infantile SMA, intermediate SMA, juvenile SMA or adult-onset SMA.

In a further embodiment, said gene coding said SMN protein is under the control of a promoter functional in lower motor neurons or spinal cord glial cells.

In another embodiment, the rAAV vector, in particular a rAAV vector comprising an AAV9 or AAVrh10 capsid, in particular an AAV9 capsid, contains a single-stranded genome comprising, in this order: an AAV 5′-ITR (such as an AAV2 5′-ITR), a promoter (such as an ubiquitous promoter, in particular the CAG promoter), a gene encoding a SMN protein (such as the human SMN1 gene), a polyadenylation signal (such as the HBB2 polyadenylation signal) and an AAV 3′-ITR (such as an AAV2 3′-ITR). In a particular embodiment, the rAAV vector comprises:
an AAV9 capsid or AAVrh10 capsid, in particular an AAV9 capsid; and
a single-stranded genome comprising, in this order: an AAV2 5′-ITR, the CAG promoter, a human SMN1 gene, a HBB2 polyadenylation signal and an AAV2 3′-ITR.

According to another aspect, the invention relates to a rAAV vector comprising
(i) an AAV9 capsid or an AAVrh10 capsid, in particular an AAV9 capsid; and
(ii) a single-stranded genome including a gene coding a spinal motor neuron (SMN) protein.

In a particular embodiment, said rAAV vector contains a genome comprising, in this order: an

AAV 5′-ITR (such as an AAV2 5′-ITR), a promoter (such as an ubiquitous promoter, in particular the CAG promoter), a gene encoding a SMN protein (such as the human SMN1 gene), a polyadenylation signal (such as the HBB2 polyadenylation signal) and an AAV 3′-ITR (such as an AAV2 3′-ITR). In another embodiment, the rAAV vector comprises:
an AAV9 capsid or AAVrh10 capsid, in particular an AAV9 capsid; and
a single-stranded genome comprising, in this order: an AAV2 5′-ITR, the CAG promoter, a human SMN1 gene, a HBB2 polyadenylation signal and an AAV2 3′-ITR.

According to a another aspect, the invention relates to an isolated nucleic acid comprising, in this order: an AAV 5′-ITR (such as an AAV2 5′-ITR), a promoter (such as an ubiquitous promoter, in particular the CAG promoter), a gene encoding a SMN protein (such as the human SMN1 gene), a polyadenylation signal (such as the HBB2 polyadenylation signal) and an AAV 3′-ITR (such as an AAV2 3′-ITR), wherein said isolated nucleic acid is configured to form a single-stranded AAV vector. According to a particular embodiment, the isolated nucleic acid comprises, in this order: an AAV2 5′-ITR, the CAG promoter, a human SMN1 gene, a HBB2 polyadenylation signal and an AAV2 3′-ITR. In a particular embodiment, said nucleic acid sequence has the sequence shown in SEQ ID NO:1 or a sequence that is at least 80% identical to SEQ ID NO:1, e.g. at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to SEQ ID NO:1.

According to another aspect, the invention relates to a plasmid comprising the isolated nucleic acid construct of the invention.

### BRIEF DESCRIPTION OF THE DRAWINGS

FIG. 1: Kaplan-Meyer survival curve of SMA mice (Smn2B/−) treated by ICV administration of ssAAV9-hSMN1 vector (n=10) compared to untreated mutant (n=4) and WT (n=10) mice.

FIG. 2: Kaplan-Meyer survival curve of untreated and ssAAV-hSMN1 treated Smn^{2B/-} mice and wild-type animals (n=10 mice per group).

### DETAILED DESCRIPTION OF THE INVENTION

The present invention provides materials and methods useful for the treatment of SMA.

More specifically, the present invention provides a recombinant adeno-associated virus (rAAV) vector comprising (i) an AAV9 capsid or an AAVrh10 capsid, and (ii) a single-stranded genome including a gene coding for a spinal motor neuron (SMN) protein, for use in a method for the treatment of spinal muscular atrophy (SMA). In a particular embodiment, the invention relates to a recombinant adeno-associated virus (rAAV) vector comprising (i) an AAV9 capsid or an AAVrh10 capsid; and (ii) a single-stranded genome including a transgene expression cassette including a gene coding a spinal motor neuron (SMN) protein, wherein said transgene expression cassette has a size comprised between 2100 nucleotides and 4400 nucleotides for use in a method for the treatment of spinal muscular atrophy (SMA).The invention further relates to a method for the treatment of SMA, comprising administering to a subject in need thereof a rAAV vector comprising (i) an AAV9 capsid or an AAVrh10 capsid, and (ii) a single-stranded genome including a gene coding for a spinal motor neuron (SMN) protein. According to another aspect, the invention relates to the use of a recombinant adeno-associated virus (rAAV) vector comprising (i) an AAV9 capsid or an AAVrh10 capsid, and (ii) a single-stranded genome including a gene coding for a spinal motor neuron (SMN) protein, for the manufacture of a medicament for the treatment of SMA. As shown in the experimental part of the present application, thanks to the invention it is possible to increase the survival of a subject having SMA at a level at least equivalent, or almost equivalent, or at a better level than the increase in survival observed using a self-complementary AAV vector. As mentioned above, this effect totally was totally unexpected since the main opinion in the field of virus-mediated gene therapy was that scAAV vectors achieve greater transduction efficiency as well as faster and more robust transgene expression. According to this general view, it was expected that this increase in transduction efficiency with self-complementary AAV vectors would translate to a significantly lower dose of vector administered to achieve the same level of gene product or number of transduced cells, as compared with traditional single stranded AAV vectors (McCarty et al., 2001, op. cit.). Against this view, it is herein shown that injection of 8×10¹² vg/kg of a ssAAV9-hSMN1 vector leads to a significant rescue of lifespan and growth of an animal model of SMA, with 40% of animals alive at 245 days. A previous study by others have shown that ICV injection of 2.6×10¹³ and 1.8×10¹³ vg/kg of a self-complementary AAV9 vector led to an increase in survival of up to 274 days and 165 days, respectively (Meyer et al., Molecular Therapy, 2015, 23(3): 477-487). Markedly, these doses are lower than the dose shown to be efficient with the single stranded AAV9 vector used by the present inventors. Strikingly, Meyer et al. did not observe a statistical difference between untreated control mice and mice treated with 1×10¹³ and 2.7×10¹² vg/kg of their self-complementary AAV9 vector, with a survival of 24 and 19 days of treated mice, respectively. From the foregoing study, it was expected that a significant survival increase of 165 days needs a dose of a self-complementary AAV9 vector (i.e. a vector known for its better transduction efficiency than a single stranded vector) injected via the ICV route of least 1.8×10¹³ vg/kg. It is herein shown that using single-stranded AAV vectors such as a single-stranded AAV9 vector may be more advantageous since both the survival rate (245 days in the present invention) and the dose implemented (8×10¹² vg/kg) are better in the present study. As shown above, nothing in the prior art suggested such a good performance for a ssAAV vector.

The present invention implements single-stranded AAV vectors, which may be advantageous as compared to self-complementary AAV vectors in that they are readily produced and in that the expression cassette that can be introduced therein may be longer. This latter point allows the possibility of introducing longer expression control sequences, and/or more expression control sequences in a single stranded AAV vector than in a self-complementary AAV vector. For a relatively small gene such as the SMN1 gene, the common view is that it is not considered as a drawback for self-complementary AAV vectors that would teach away one skilled in the art from such self-complementary AAV vectors. Quite on the contrary, as mentioned above, before the present invention, self-complementary AAV vectors were considered the vectors of choice as compared to single-stranded AAV vectors thanks to their recognized better transduction efficiency. We herein show that a rAAV containing a SMN gene may be as advantageous as a single-stranded vector, or more advantageous, than a self-complementary vector.

In the context of the present invention, a rAAV vector may comprise an AAV9 or AAVrh10 capsid. Such vector is herein termed “AAV9 vector” or “AAVrh10 vector”, respectively, independently of the serotype the genome contained in the rAAV vector is derived from. Accordingly, an AAV9 vector may be a vector comprising an AAV9 capsid and an AAV9 derived genome (i.e. comprising AAV9 ITRs) or a pseudotyped vector comprising an AAV9 capsid and a genome derived from a serotype different from the AAV9 serotype. Likewise, an

AAVrh10 vector may be a vector comprising an AAVrh10 capsid and an AAV10 derived genome (i.e. comprising AAVrh10 ITRs) or a pseudotyped vector comprising an AAVrh10 capsid and a genome derived from a serotype different from the AAVrh10 serotype.

The genome present within the rAAV vector of the present invention is single-stranded. In particular, a “single stranded genome” is a genome that is not self-complementary, i.e. the coding region contained therein has not been designed as disclosed in McCarty et al., 2001 and 2003 (Op. cit) to form an intra-molecular double-stranded DNA template.

The genome present within the rAAV vector lacks AAV rep and cap genes, and comprises a gene coding for a SMN protein flanked by AAV ITRs. The AAV ITR may be from any AAV serotype including, but not limited to, AAV1, AAV2, AAV3, AAV4, AAV5, AAV6, AAV7, AAV8, AAV9, AAVrh10 and AAV11. In a particular embodiment, the rAAV vector used according to the present invention has an AAV9 capsid and a single-stranded genome comprising 5′ and 3′ ITRs selected from AAV1, AAV2, AAV3, AAV4, AAV5, AAV6, AAV7, AAV8, AAV9, AAVrh10 and AAV11 ITRs. In a further embodiment, the rAAV vector used according to the present invention has an AAV9 capsid and a single-stranded genome comprising 5′ and 3′ AAV2 ITRs. In a further particular embodiment, the rAAV vector used according to the present invention has an AAVrh10 capsid and a single-stranded genome comprising 5′ and 3′ ITRs selected from AAV1, AAV2, AAV3, AAV4, AAV5, AAV6, AAV7, AAV8, AAV9, AAVrh10 and AAV11 ITRs. In a further embodiment, the rAAV vector used according to the present invention has an AAVrh10 capsid and a single-stranded genome comprising 5′ and 3′ AAV2 ITRs.

In a particular embodiment, the SMN protein is human SMN protein. In a particular embodiment, the nucleic acid coding the human SMN protein is derived from the sequence having the Genbank accession No. NM_000344.3. In a particular embodiment, the gene encoding the SMN protein consists of or comprises the sequence shown in SEQ ID NO:8. In another particular embodiment, the sequence of the gene encoding the SMN protein, in particular the human SMN protein, is optimized. Sequence optimization may include a number of changes in a nucleic acid sequence, including codon optimization, increase of GC content, decrease of the number of CpG islands, decrease of the number of alternative open reading frames (ARFs) and/or decrease of the number of splice donor and splice acceptor sites. Because of the degeneracy of the genetic code, different nucleic acid molecules may encode the same protein. It is also well known that the genetic codes of different organisms are often biased towards using one of the several codons that encode the same amino acid over the others. Through codon optimization, changes are introduced in a nucleotide sequence that take advantage of the codon bias existing in a given cellular context so that the resulting codon optimized nucleotide sequence is more likely to be expressed in such given cellular context at a relatively high level compared to the non-codon optimised sequence. In a preferred embodiment of the invention, such sequence optimized nucleotide sequence encoding a SMN protein is codon-optimized to improve its expression in human cells compared to non-codon optimized nucleotide sequences coding for the same SMN protein, for example by taking advantage of the human specific codon usage bias.

In a particular embodiment, the optimized SMN coding sequence is codon optimized, and/or has an increased GC content and/or has a decreased number of alternative open reading frames, and/or has a decreased number of splice donor and/or splice acceptor sites, as compared to the wild-type human SMN1 coding sequence of SEQ ID NO:8. In a particular embodiment, the nucleic acid sequence encoding the SMN protein is at least 70% identical, in particular at least 75% identical, at least 80% identical, at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the sequence shown in SEQ ID NO:8.

As mentioned above, in addition to the GC content and/or number of ARFs, sequence optimization may also comprise a decrease in the number of CpG islands in the sequence and/or a decrease in the number of splice donor and acceptor sites. Of course, as is well known to those skilled in the art, sequence optimization is a balance between all these parameters, meaning that a sequence may be considered optimized if at least one of the above parameters is improved while one or more of the other parameters is not, as long as the optimized sequence leads to an improvement of the transgene, such as an improved expression and/or a decreased immune response to the transgene in vivo.

In addition, the adaptiveness of a nucleotide sequence encoding a SMN protein to the codon usage of human cells may be expressed as codon adaptation index (CAI). A codon adaptation index is herein defined as a measurement of the relative adaptiveness of the codon usage of a gene towards the codon usage of highly expressed human genes. The relative adaptiveness (w) of each codon is the ratio of the usage of each codon, to that of the most abundant codon for the same amino acid. The CAI is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Kim et al, Gene. 1997, 199:293-301; zur Megede et al, Journal of Virology, 2000, 74: 2628-2635).

In a particular embodiment, the nucleic acid sequence coding for human SMN protein consists of or comprises an optimized sequence as sequence shown in SEQ ID NO:12, SEQ ID NO:13, SEQ ID NO:14 or SEQ ID NO:15.

In another particular embodiment, the genome of the rAAV vector comprises an expression cassette including the gene coding for the SMN protein. In the context of the present invention, an “expression cassette” or “transgene expression cassette” is a nucleic acid sequence comprising a transgene (here, a gene coding a SMN protein) operably linked to sequences allowing the expression of said transgene in an eukaryotic cell. In the AAV vectors of the present invention, the gene coding for a SMN protein may be operably linked to one or more expression control sequences and/or other sequences improving the expression of the transgene. As used herein, the term “operably linked” refers to a linkage of polynucleotide elements in a functional relationship. A nucleic acid is “operably linked” when it is placed into a functional relationship with another nucleic acid sequence. For instance, a promoter, or another transcription regulatory sequence, is operably linked to a coding sequence if it affects the transcription of the coding sequence. Such expression control sequences are known in the art, such as promoters, enhancers, introns, polyadenylation signals, etc.

In a particular embodiment, the expression cassette has a size comprised between 2100 and 4400 nucleotides, in particular between 2700 and 4300 nucleotides, more particularly between 3200 and 4200 nucleotides. In a particular embodiment, the size of the expression cassette is of about 3200 nucleotides, about 3300 nucleotides, about 3400 nucleotides, about 3500 nucleotides, about 3600 nucleotides, about 3700 nucleotides, about 3800 nucleotides, about 3900 nucleotides, about 4000 nucleotides, about 4100 nucleotides, or about 4200 nucleotides. According to the present invention, the term “about”, when referring to a numerical value, means plus or minus 5% of this numerical value.

In the rAAV vector of the invention, the gene coding for a SMN protein is operably linked to a promoter.

According to the invention, the promoter is functional at least in lower motor neurons or spinal cord glial cells, preferably at least in lower motor neurons. Promoters functional in motor neurons include, without limitation, ubiquitous and motor neuron-specific promoters. Representative ubiquitous promoters include the cytomegalovirus enhancer/chicken beta actin promoter, first exon and first intron/splice acceptor of the rabbit beta-globin gene (i.e. the CAG promoter resulting from the fusion of the sequences shown in SEQ ID NO:3, 4, 5 and 6, in this order from 5′ to 3′), the cytomegalovirus enhancer/promoter (CMV) (optionally with the CMV enhancer) [see, e.g., Boshart et al, Cell, 41:521-530 (1985)], the SV40 early promoter, the retroviral Rous sarcoma virus (RSV) LTR promoter (optionally with the RSV enhancer), the dihydrofolate reductase promoter, the β-actin promoter, and the EF1 promoter. Representative promoters specific for the motor neurons include the promoter of the Calcitonin Gene-Related Peptide (CGRP), a known motor neuron-derived factor. Other promoters functional in motor neurons include the promoters of Choline Acetyl Transferase (ChAT) , Neuron Specific Enolase (NSE), Synapsin, Hb9 or ubiquitous promoters including Neuron-Restrictive Silencer Elements (NRSE). Representative promoters specific for glial cells include the promoter of the Glial Fibrillary Acidic Protein gene (GFAP).

The expression cassette may further comprise a polyadenylation signal. Illustrative polyadenylation signals include, without limitation, the SMN1 gene polyadenylation signal, or a heterologous polyadenylation signal such as the human beta globin (HBB2) polyadenylation signal (such as the sequence shown in SEQ ID NO:9 or SEQ ID NO:16), the bovine growth hormone polyadenylation signal, the SV40 polyadenylation signal, or another naturally occurring or artificial polyadenylation signal.

Other sequences such as a Kozak sequence (such as that shown in SEQ ID NO:7) are known to those skilled in the art and are introduced to allow expression of a transgene.

In a particular embodiment, the expression cassette comprises, in this order: a promoter, a gene encoding a SMN protein and a polyadenylation signal.

In another particular embodiment, the expression cassette may comprise a further regulatory element located between the gene encoding a SMN protein and the polyadenylation signal.

Representative regulatory elements that may be useful in the present invention include, without limitation, the 3′-untranslated region (3′-UTR) of a gene, such as the 3′-UTR of the gene encoding a SMN protein (such as the 3′-UTR of the human SMN1 gene, for example the sequence shown in SEQ ID NO:17), the 3′-UTR of the HBB2 gene, the 3′-UTR of SV40 or the 3′-UTR of the bovine growth hormone.

In a particular embodiment, the expression cassette does not comprise a WPRE sequence, such as the WPRE shown in SEQ ID NO:18.

In another embodiment, the expression cassette comprises, in this order: the CAG promoter (e.g. the sequence resulting from the fusion of the sequences shown in SEQ ID NO:3, 4, 5 and 6, in this order from 5′ to 3′), a gene encoding a SMN protein (such as the sequence shown in SEQ ID NO:8, SEQ ID NO:12, SEQ ID NO:13, SEQ ID NO:14 or SEQ ID NO:15, in particular SEQ ID NO:8) and a polyadenylation signal (such as a HBB2 polyadenylation signal, such as the sequence shown in SEQ ID NO:9 or SEQ ID NO:17, in particular SEQ ID NO:9).

In a particular embodiment, the genome in the rAAV vector of the invention (in particular a rAAV9 vector of the invention) comprises, in this order: an AAV 5′-ITR (such as an AAV2 5′-ITR, in particular the sequence shown in SEQ ID NO:2), a promoter (such as an ubiquitous promoter, in particular the CAG promoter), a gene encoding a SMN protein (such as the human SMN protein), a polyadenylation signal (such as the HBB2 polyadenylation signal) and an AAV 3′-ITR (such as an AAV2 3′-ITR, in particular the sequence shown in SEQ ID NO:10).

In a further particular embodiment, the genome of the rAAV vector comprises, in this order: an AAV2 5′-ITR (such as the sequence shown in SEQ ID NO:2), the CAG promoter, a human SMN1 gene, a HBB2 polyadenylation signal and an AAV2 3′-ITR (such as the sequence shown in SEQ ID NO:10). In particular, the genome comprises the sequence shown in SEQ ID NO:1.

In another particular embodiment, the genome comprises a nucleic acid sequence allowing the expression of a SMN protein in an eukaryotic cell that is at least 80% identical to SEQ ID NO:1, e.g. at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to SEQ ID NO:1. In another particular embodiment, the genome comprises a nucleic acid sequence allowing the expression of a SMN protein in an eukaryotic cell that is at least 80% identical to SEQ ID NO:11, e.g. at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to SEQ ID NO:11.

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:8;
a HBB2 polyadenylation signal, such as the sequence shown in SEQ ID NO:9 or SEQ ID NO:16; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:12;
a HBB2 polyadenylation signal, such as the sequence shown in SEQ ID NO:9 or SEQ ID NO:16; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:13;
a HBB2 polyadenylation signal, such as the sequence shown in SEQ ID NO:9 or SEQ ID NO:16; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:14;
a HBB2 polyadenylation signal, such as the sequence shown in SEQ ID NO:9 or SEQ ID NO:16; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:15;
a HBB2 polyadenylation signal, such as the sequence shown in SEQ ID NO:9 or SEQ ID NO:16; and
an AAV2 3′-ITR (such as the sequence shown in SEQ ID NO:10). In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:8;
the HBB2 polyadenylation signal of SEQ ID NO:9; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:12;
the HBB2 polyadenylation signal of SEQ ID NO:9; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:13;
the HBB2 polyadenylation signal of SEQ ID NO:9; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:14;
the HBB2 polyadenylation signal of SEQ ID NO:9; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:15;
the HBB2 polyadenylation signal of SEQ ID NO:9; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:8;
a regulatory element, such as the human SMN1 3′-UTR (e.g. the sequence shown in SEQ ID NO:17);
a HBB2 polyadenylation signal, such as the sequence shown in SEQ ID NO:9 or SEQ ID NO:16; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:12;
a regulatory element, such as the human SMN1 3′-UTR (e.g. the sequence shown in SEQ ID NO:17);
a HBB2 polyadenylation signal, such as the sequence shown in SEQ ID NO:9 or SEQ ID NO:16; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:13;
a regulatory element, such as the human SMN1 3′-UTR (e.g. the sequence shown in SEQ ID NO:17);
a HBB2 polyadenylation signal, such as the sequence shown in SEQ ID NO:9 or SEQ ID NO:16; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:14;
a regulatory element, such as the human SMN1 3′-UTR (e.g. the sequence shown in SEQ ID NO:17);
a HBB2 polyadenylation signal, such as the sequence shown in SEQ ID NO:9 or SEQ ID NO:16; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:15;
a regulatory element, such as the human SMN1 3′-UTR (e.g. the sequence shown in SEQ ID NO:17);
a HBB2 polyadenylation signal, such as the sequence shown in SEQ ID NO:9 or SEQ ID NO:16; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:8;
a regulatory element, such as the human SMN1 3′-UTR (e.g. the sequence shown in SEQ ID NO:17);
the HBB2 polyadenylation signal of SEQ ID NO:16; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:12;
a regulatory element, such as the human SMN1 3′-UTR (e.g. the sequence shown in SEQ ID NO:17);
the HBB2 polyadenylation signal of SEQ ID NO:16; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:13;
a regulatory element, such as the human SMN1 3′-UTR (e.g. the sequence shown in SEQ ID NO:17);
the HBB2 polyadenylation signal of SEQ ID NO:16; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:14;
a regulatory element, such as the human SMN1 3′-UTR (e.g. the sequence shown in SEQ ID NO:17);
the HBB2 polyadenylation signal of SEQ ID NO:16; and
an AAV2 3′-ITR (such as the sequence shown in SEQ ID NO:10).

In another particular embodiment, the genome of the rAAV vector comprises, in this order:
an AAV2 5′-ITR (such as the sequence shown in SEQ ID NO:2),
the CAG promoter,
a human SMN1 coding sequence consisting of SEQ ID NO:15;
a regulatory element, such as the human SMN1 3′-UTR (e.g. the sequence shown in SEQ ID NO:17);
the HBB2 polyadenylation signal of SEQ ID NO:16; and
an AAV2 3′-ITR (such as the sequence shown in SEQ ID NO:10).

Thanks to the present invention, the gene coding for the SMN protein may be delivered to lower motor neurons, such as to spinal cord motor neurons (i.e. motor neurons whose soma is within the spinal cord) and to spinal cord glial cells.

In a particular embodiment, SMA is neonatal SMA, infantile SMA, intermediate SMA, juvenile SMA or adult-onset SMA.

In another aspect, the invention provides DNA plasmids comprising rAAV genomes of the invention. Production of rAAV requires that the following components are present within a single cell (denoted herein as a packaging cell): a rAAV genome, AAV rep and cap genes separate from (i.e., not in) the rAAV genome, and helper virus functions. Production of pseudotyped rAAV is disclosed in, for example, WO 01/83692. Production may implement transfection a cell with two, three or more plasmids. For example three plasmids may be used, including: (i) a plasmid carrying a Rep/Cap cassette, (ii) a plasmid carrying the rAAV genome (i.e. a transgene flanked with AAV ITRs) and (iii) a plasmid carrying helper virus functions (such as adenovirus helper functions). In another embodiment, a two-plasmid system may be used, comprising (i) a plasmid comprising Rep and Cap genes, and helper virus functions, and (ii) a plasmid comprising the rAAV genome.

Accordingly, in a particular aspect, the invention further relates to an isolated nucleic acid comprising, in this order: an AAV 5′-ITR (such as an AAV2 5′-ITR, in particular the sequence shown in SEQ ID NO:2), an expression cassette as defined above, according to any embodiment provided above, and an AAV ITR. In a particular embodiment, the isolated nucleic acid of the invention comprises, in this order: a promoter (such as an ubiquitous promoter, in particular the CAG promoter), a gene encoding a SMN protein (such as the human SMN1 gene), a polyadenylation signal (such as the HBB2 polyadenylation signal) and an AAV 3′-ITR (such as an AAV2 3′-ITR, in particular the sequence shown in SEQ ID NO:10), wherein said isolated nucleic acid is configured to form a single-stranded AAV vector. In particular, the isolated nucleic acid of the invention may comprise, in this order: an AAV2 ITR, the CAG promoter, a human SMN gene (such as the human SMN1 gene), a HBB2 polyadenylation signal and an AAV2 ITR. More particularly, the isolated nucleic acid of the invention may comprise a nucleic acid sequence allowing the expression of the SMN protein in an eukaryotic cell that is at least 80% identical to SEQ ID NO:1, e.g. at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to SEQ ID NO:1. More particularly, the isolated nucleic acid of the invention may comprise a nucleic acid sequence allowing the expression of the SMN protein in an eukaryotic cell that is at least 80% identical to SEQ ID NO:11, e.g. at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to SEQ ID NO:11.

In a further aspect, the invention relates to a plasmid comprising the isolated nucleic acid construct of the invention. This plasmid may be introduced in a cell for producing a rAAV vector according to the invention by providing the rAAV genome to said cell.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are incorporated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial, and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62: 1963 (1988); and Lebkowski et al., 1988 Mol. Cell. Biol., 7:349 (1988); Samulski et al. (1989, J. Virol., 63:3822-3828); U.S. Pat. No. 5,173,414; WO 95/13365 and corresponding U.S. Pat. No. 5,658.776 ; WO 95/13392; WO 96/17947; PCT/U598/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13: 1244-1250; Paul et al. (1993) Human Gene Therapy 4:609-615; Clark et al. (1996) Gene Therapy 3: 1124-1132; U.S. Pat. Nos. 5,786,211; 5,871,982; and 6,258,595. The invention thus also provides packaging cells that produce infectious rAAV. In one embodiment packaging cells may be stably transformed cancer cells such as HeLa cells, HEK293 cells, HEK 293T, HEK293vc and PerC.6 cells (a cognate 293 line). In another embodiment, packaging cells are cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

The rAAV may be purified by methods standard in the art such as by column chromatography or cesium chloride gradients. Methods for purifying rAAV vectors from helper virus are known in the art and include methods disclosed in, for example, Clark et ah, Hum. Gene Ther., 10(6): 1031-1039 (1999); Schenpp and Clark, Methods Mol. Med., 69: 427-443 (2002); U.S. Pat. No. 6,566,118 and WO 98/09657.

In another aspect, the invention provides compositions comprising a rAAV disclosed in the present application. Compositions of the invention comprise rAAV in a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

The rAAV vector for use according to the invention may be administered locally with or without systemic co-delivery. In the context of the present invention, local administration denotes an administration into the cerebrospinal fluid of the subject, such as via an intrathecal injection of the rAAV vector. In some embodiment, the methods further comprise administrating an effective amount of rAAV by intracerebral administration. In some embodiment, the rAAV may be administrated by intrathecal administration and by intracerebral administration. In some embodiment the rAAV vector may be administrated by a combined intrathecal and/or intracerebral and/or peripheral (such as a vascular, for example intravenous or intra-arterial, in particular intravenous) administration.

As used herein the term “intrathecal administration” refers to the administration of a rAAV or a. composition comprising a rAAV, into the spinal canal. For example, intrathecal administration may comprise injection in the cervical region of the spinal canal, in the thoracic region of the spinal canal, or in the lumbar region of the spinal canal. Typically, intrathecal administration is performed by injecting an agent, e.g., a composition comprising a rAAV, into the subarachnoid cavity (subarachnoid space) of the spinal canal, which is the region between the arachnoid membrane and pia mater of the spinal canal. The subarchnoid space is occupied by spongy tissue consisting of trabeculae (delicate connective tissue filaments that extend from the arachnoid mater and blend into the pia mater) and intercommunicating channels in which the cerebrospinal fluid is contained. In some embodiments, intrathecal administration is not administration into the spinal vasculature. In certain embodiment the intrathecal administration is in the lumbar region of the subject

As used herein, the term “intracerebral administration” refers to administration of an agent into and/or around the brain. Intracerebral administration includes, but is not limited to, administration of an agent into the cerebrum, medulla, pons, cerebellum, intracranial cavity, and meninges surrounding the brain. Intracerebral administration may include administration into the dura mater, arachnoid mater, and pia mater of the brain. Intracerebral administration may include, in some embodiments, administration of an agent into the cerebrospinal fluid (CSF) of the subarachnoid space surrounding the brain. Intracerebral administration may include, in some embodiments, administration of an agent into ventricles of the brain/forebrain, e.g., the right lateral ventricle, the left lateral ventricle, the third ventricle, the fourth ventricle. In some embodiments, intracerebral administration is not administration into the brain vasculature.

In some embodiments, intracerebral administration involves injection using stereotaxic procedures. Stereotaxic procedures are well known in the art and typically involve the use of a computer and a 3-dimensional scanning device that are used together to guide injection to a particular intracerebral region, e.g., a ventricular region. Micro-injection pumps (e.g., from World Precision Instruments) may also be used. In some embodiments, a microinjection pump is used to deliver a composition comprising a rAAV. In some embodiments, the infusion rate of the composition is in a range of 1 μl/minute to 100 μl/minute. As will be appreciated by the skilled artisan, infusion rates will depend on a variety of factors, including, for example, species of the subject, age of the subject, weight/size of the subject, serotype of the rAAV, dosage required, intracerebral region targeted, etc. Thus, other infusion rates may be deemed by a skilled artisan to be appropriate in certain circumstances.

Furthermore, thanks to its capacity to cross the blood-brain barrier, the rAAV vector implemented in the invention (i.e. rAAV9 or rAAVrh10 vector) may be administered via a systemic route. Accordingly, methods of administration of the rAAV vector include but are not limited to, intramuscular, intraperitoneal, vascular (e.g. intravenous or intra-arterial), subcutaneous, intranasal, epidural, and oral routes. In a particular embodiment, the systemic administration is a vascular injection of the rAAV vector, in particular an intravenous injection.

In a particular embodiment, the rAAV vector is administered into the cerebrospinal fluid, in particular by intrathecal injection. In a particular embodiment, the patient is put in the Trendelenberg position after intrathecal delivery of the rAAV vector.

The amount of the rAAV vector of the invention which will be effective in the treatment of SMA can be determined by standard clinical techniques. In addition, in vivo and/or in vitro assays may optionally be employed to help predict optimal dosage ranges. The dosage of the rAAV vector of the invention administered to the subject in need thereof will vary based on several factors including, without limitation, the specific type or stage of the disease treated, the subject's age or the level of expression necessary to obtain the therapeutic effect. One skilled in the art can readily determine, based on its knowledge in this field, the dosage range required based on these factors and others. Typical doses of the vector are of at least 1×10⁸ vector genomes per kilogram body weight (vg/kg), such as at least 1×10⁹ vg/kg, at least 1×10¹⁰ vg/kg, at least 1×10¹¹ vg/kg, at least 1×10¹² vg/kg at least 1×10¹³ vg/kg, at least 1×10¹⁴ vg/kg or at least 1×10¹⁵ vg/kg.

According to another aspect, the invention relates to a rAAV vector comprising
(i) an AAV9 capsid or an AAVrh10 capsid, in particular an AAV9 capsid; and
(ii) a single-stranded genome including a gene coding a spinal motor neuron (SMN) protein.

In a particular embodiment, the single-genome comprises a CAG promoter. In particular, the genome comprises in this order: an AAV 5′-ITR, a CAG promoter, the gene coding a SMN protein, a polyadenylation signal and an AAV 3′-ITR.

In another embodiment, the single-genome comprises a HBB2 polyadenylation signal. In particular, the genome comprises, in this order: an AAV 5′-ITR, a promoter, the gene coding a SMN protein, a HBB2 polyadenylation signal and an AAV 3′-ITR.

In these embodiments, the single-stranded genome may further comprise a further regulatory element such as a 3′-UTR of a gene, such as the 3′-UTR of the SMN1 gene, between the gene and the polyadenylation signal.

In a particular embodiment, the genome of the rAAV vector comprises, in this order:
an AAV 5′-ITR, such as an AAV2 5′-ITR (such as the sequence shown in SEQ ID NO:2),
an expression cassette as defined above, according to any embodiment provided above,
an AAV 3′-ITR, such as an AAV2 3′-ITR (such as the sequence shown in SEQ ID NO:10).

In a further particular embodiment, the genome of the rAAV vector comprises, in this order: an AAV2 5′-ITR, the CAG promoter, a human SMN1 gene, a HBB2 polyadenylation signal and an AAV2 3′-ITR. In particular, the genome comprises the sequence shown in SEQ ID NO:1, or a sequence allowing the expression of a SMN protein in an eukaryotic cell and that is at least 80% identical to SEQ ID NO:1, e.g. at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to SEQ ID NO:1. In particular, the genome comprises the sequence shown in SEQ ID NO:1, or a sequence allowing the expression of a SMN protein in an eukaryotic cell and that is at least 80% identical to SEQ ID NO:1, e.g. at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to SEQ ID NO:11.

### Particular Objects of the Invention

1. An isolated nucleic acid sequence comprising, in this order:
an AAV 5′-ITR;
a promoter;
a gene encoding a SMN protein;
optionally, a further regulatory element which is not a WPRE;
a polyadenylation signal; and
an AAV 3′-ITR;
   wherein said isolated nucleic acid is configured to form a single-stranded AAV genome which is not self-complementary.

2. The isolated nucleic acid sequence according to object 1, the AAV 5′-ITR is an AAV2 5′-ITR and the AAV 3′-ITR is an AAV2 3′-ITR.

3. The isolated nucleic acid sequence according to object 1 or 2, wherein the promoter is an ubiquitous promoter.

4. The isolated nucleic acid sequence according to object 3, wherein the ubiquitous promoter is a CAG promoter.

5. The isolated nucleic acid sequence according to any one of objects 1 to 4, wherein the gene encoding a SMN protein is the human SMN1 gene.

6. The isolated nucleic acid sequence according to any one of objects 1 to 5, wherein said nucleic acid sequence does not comprise a SV40 intron between the promoter and the gene.

7. The isolated nucleic acid sequence according to any one of objects 1 to 6, wherein said polyadenylation signal is not a polyadenylation signal sequence from bovine growth hormone.

8. The isolated nucleic acid sequence according to any one of objects 1 to 7, wherein the polyadenylation signal is the HBB2 polyadenylation signal.

9. The isolated nucleic acid sequence according to any one of objects 1 to 8, comprising, in this order:
an AAV2 5′-ITR;
the CAG promoter;
a human SMN1 gene;
optionally, a further regulatory element which is not a WPRE;
a HBB2 polyadenylation signal; and
an AAV2 3′-ITR.

10. The isolated nucleic acid sequence according to any one of objects 1 to 9, wherein the further regulatory element is the 3′-untranslated region (UTR) of the gene encoding a SMN protein.

11. The isolated nucleic acid sequence according to object 10, wherein the further regulatory element is the 3′-UTR of the human SMN1 gene.

12. The isolated nucleic acid sequence according to any one of objects 1 to 11, comprising, in this order:
an AAV2 5′-ITR,
the CAG promoter,
a human SMN1 gene,
the 3′-UTR of the human SMN1 gene,
a HBB2 polyadenylation signal; and
an AAV2 3′-ITR.

13. The isolated nucleic acid sequence according to any one of objects 1 to 12, wherein said nucleic acid sequence comprises or consists of the sequence shown in SEQ ID NO:1 or SEQ ID NO:11, or a sequence that is at least 80% identical to SEQ ID NO:1, e.g. at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to SEQ ID NO:1 or SEQ ID NO:11.

14. A vector that comprises the nucleic acid sequence according to any one of objects 1 to 13.

15. The vector according to object 14, which is a plasmid or an AAV vector.

16. The vector according to object 15, wherein the AAV vector comprises a capsid selected from an AAV9 capsid and an AAVrh10 capsid.

17. The vector according to object 15 or 16, wherein the AAV vector comprises an AAV9 capsid.

18. The vector according to any one of claims 14 to 17, for use in a method for the treatment of spinal muscular atrophy (SMA).

19. The vector for use according to claim 18, wherein said vector is for administration into the cerebrospinal fluid of a subject.

20. The vector for use according to claim 19, wherein said vector is for administration by intrathecal and/or intracerebroventricular injection.

### EXAMPLES

### Example 1

It is herein demonstrated that survival of a mouse model of SMA is greatly improved, beyond expectation, after administration of an AAV vector carrying a human SMN1 gene into a single-stranded genome.

Materials and Methods

Vector Production

The AAV vector used is a single-stranded recombinant AAV9 vector carrying human SMN1 gene under the control of the CAG promoter (a hybrid CMV enhancer/chicken-β-actin promoter and beta-globin splice acceptor site), and a polyA region from the HBB2 gene.

The ssAAV9 vector was produced by the tri-transfection system using standard procedures (Xiao et al., J. Virol. 1998; 72:2224-2232). Pseudo-typed recombinant rAAV2/9 (rAAV9) viral preparations were generated by packaging AAV2-inverted terminal repeat (ITR) recombinant genomes into AAV9 capsids. Briefly, the cis-acting plasmid carrying the CAG-hSMN1 construct, a trans-complementing rep-cap9 plasmid encoding the proteins necessary for the replication and structure of the vector and an adenovirus helper plasmid were co-transfected into HEK293 cells. Vector particles were purified through two sequential cesium chloride gradient ultra-centrifugations and dialyzed against sterile PBS-MK. DNAse I resistant viral particles were treated with proteinase K. Viral titres were quantified by a TaqMan real-time PCR assay (Applied Biosystem) with primers and probes specific for the polyA region and expressed as viral genomes per ml (vg/ml).

Animals

Smn^{2B/-} mice were obtained by two colonies crossing Smn^{2B/2B} homozygous (kindly provided by Rashmi Kothary, Ottawa, Ontario, Canada) and Smn^{+/-} heterozygous mice (Jackson Laboratories) were mated to generate Smn^{2B/+} and Smn^{2B/-} mice. Litters were genotyped at birth. Mice were kept under a 12-hour light 12-hour dark cycle and fed with a standard diet supplemented with Diet Recovery gel, food and water ad libitum. Care and manipulation of mice were performed in accordance with national and European legislations on animal experimentation and approved by the institutional ethical committee.

In Vivo Gene Therapy

Smn^{2B/-} mice were treated with viral particles at birth (P0) by intracerebroventricular (ICV) injections; ssAAV9-hSMN1 (8×10e¹² vg/kg, 7 μl total volume) was administrated into the right lateral ventricle. Control Smn^{2B/+} littermates and wild-type mice received 7 pl of PBS-MK (1 mM MgCl₂, 2.5 mM KCI) at birth using the same procedure.

Results

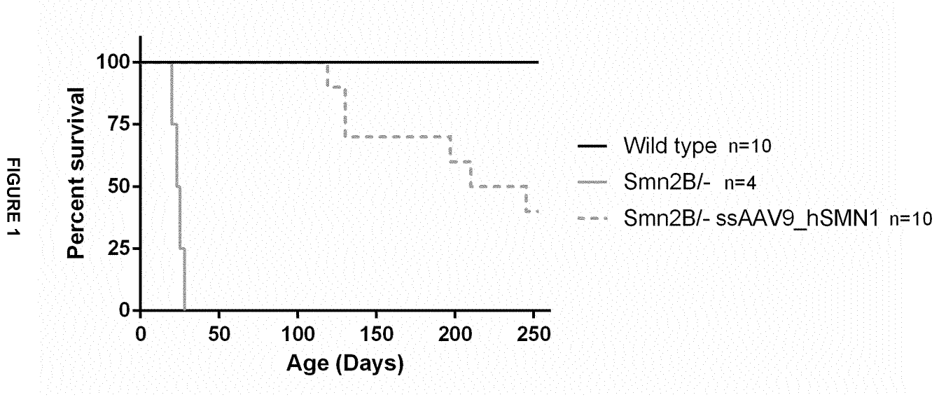

The results are presented in FIG. 1.

It can be seen that all non-treated Smn^{2B/-} mice died at around 25 days of age (n=4). On the contrary, at the end of the study, i.e. at day 200, 70% of the treated mice (n=10) were still alive, showing the impressive survival improvement obtained thanks to the rAAV vector of the present invention. Impressively, at day 245 40% of animals were still alive.

Previously, the SMNΔ7 mouse model was used for the assessment of AAV9 gene therapy of SMA, a model that presents a more severe phenotype than the Smn^{2B/-} mouse model, and that did not allow to observe this long term improvement because of early death of said SMNΔ7 mice. Although previous data showed that the AAV9 capsid was responsible for an AAV9 vector to cross the blood-brain barrier and to transduce motor neurons and glial cells in the central nervous system, the common general knowledge in this field would have incited those skilled in the art to implement double-stranded self-complementary AAV9 vectors rather than single-stranded AAV9 vectors to obtain optimal survival improvement. An improvement to the extent presented herein was therefore unexpected.

### Example 2

Additional experiments were conducted to show the improvements obtained with the present invention.

The aim of the study is to assess the therapeutic efficacy of single-stranded (ss)AAV9 vectors that express human SMN1 in a mouse model of spinal muscular atrophy. We compared the effect of three ssAAV9-hSMN1 vectors and one ssAAVrh10-hSMN1 vector by intracerebroventricular (ICV) administration in Smn^{2B/-} newborn mice 21 and 90 days post-injection.

We analyzed different parameters:
Survival,
Body weight,
Locomotion and muscle strength,
Vector biodistribution and transgene expression,
Human SMN protein expression in various tissues,
Spinal motor neuron counting,
Skeletal muscle histology,
Neuromuscular Junction (NMJ) morphology.

Three ssAAV9-hSMN1 vectors (7209, 7210 and 7211) containing the wild-type human SMN1 coding sequence (NCBI Reference Sequence: NM_000344.3) and different promoters and regulatory sequences were produced by the tri-transfection system in HEK293 cells. The vectors are designed as indicated below:

Vector 7209:

plasmid carrying the CAG promoter, human SMN1 gene, human SMN1 3′-UTR and a polyA region from the HBB2 gene;

Vector 7210:

the vector of example 1, carrying the CAG promoter, human SMN1 gene, and a polyA region from the HBB2 gene;

Vector 7211:

vector carrying the CAG promoter, human SMN1 gene, a Woodchuck hepatitis virus posttranscriptional regulatory element (WPRE) and a polyA region from the HBB2 gene.

The ssAAVrh10-hSMN1 vector was also produced by the tri-transfection system in HEK293 cells. It contains the following elements: the CAG promoter, human SMN1 gene, and a polyA region from the HBB2 gene.

We administered the vectors into the cerebrospinal fluid of Smn^{2B/-} newborn mice (post-natal day 0-1-P0/1 by ICV injection). Smn^{2B/-} mice develop a severe phenotype with body weight loss and clinical signs of the disease at around 15 days of age; the current mean survival of Smn^{2B/-} mice of our colony is 26 days (mouse line developed by Bowermann et al. Neuromusc Disord 2012 March; 22(3):263-76).

In vivo protocols were designed to assess the lifespan of mutant mice after treatment compared to controls. A group of animals (serie 3, n=10 mice per group) was used to analyze the life expectancy of treated Smn2B/− mice compared to uninjected mutant mice.

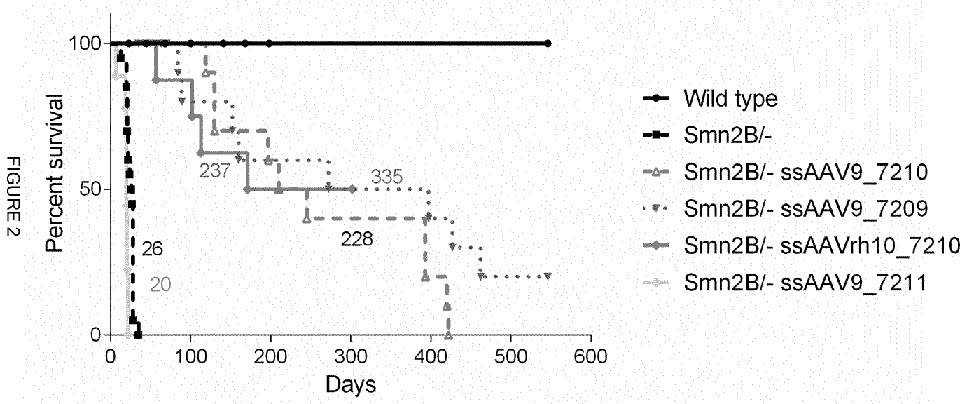

To date there are four ongoing in vivo protocols to assess the effect of ssAAV9_7209, _7210, and _7211 vectors, and ssAAVrh10_7210. The dose used in these experiments was 8×10¹² vg/Kg for all vectors. FIG. 2 shows the survival rate of treated and untreated Smn^{2B/-} mice and wild-type animals, with a clear prolongation of lifespan after treatment (the mean survival at the time of data collection for each vector is indicated in the graph).

1-45 (canceled)

46. A rAAV vector comprising:
(a)(i) an AAV9 capsid or an AAVrh10 capsid; and
(a)(ii) a single-stranded genome which is not self-complementary, said genome comprising a gene coding a human survival motor neuron (SMN) protein under the control of a CAG promoter; or
(b)(i) a capsid selected from an AAV9 capsid and an AAVrh10 capsid; and
(b)(ii) a single-stranded genome which is not self-complementary, said genome containing a nucleic acid sequence comprising, in this order:
an AAV 5′-ITR;
a CAG promoter;
a gene coding a human survival of motor neuron (SMN) protein;
optionally, a further regulatory element;
a HBB2 polyadenylation signal; and
an AAV 3′-ITR;
wherein the further regulatory element is not a WPRE; or
(c)(i) an AAV9 capsid or an AAVrh10 capsid; and
(c)(ii) a single-stranded genome which is not a self-complementary genome including a gene coding a human survival motor neuron (SMN) protein, wherein said genome does not comprise a WPRE and wherein said genome does not comprise a SV40 intron.

47. The rAAV vector according to claim 46, the genome of subsection (a) comprising, in this order: an AAV 5′-ITR, a promoter, a gene encoding a SMN protein, a polyadenylation signal and an AAV 3′-ITR.

48. The rAAV vector according to claim 47, wherein the AAV 5′-ITR is an AAV2 5′-ITR and the AAV 3′-ITR is an AAV2 3′-ITR.

49. The rAAV vector according to claim 47, wherein the gene encoding a SMN protein is the human SMN1 gene.

50. The rAAV vector according to claim 47, wherein the polyadenylation signal is the HBB2 polyadenylation signal.

51. The rAAV vector according to claim 47, wherein said rAAV vector comprises:
(i) an AAV9 capsid or an AAVrh10 capsid; and
(ii) a single-stranded genome comprising, in this order: an AAV2 5′-ITR, the CAG promoter, a human SMN1 gene, a HBB2 polyadenylation signal and an AAV2 3′-ITR.

52. The rAAV vector according to claim 47, wherein the genome comprises a further regulatory element located between the gene encoding a human SMN protein and the polyadenylation signal.

53. The rAAV vector according to claim 52, wherein the further regulatory element corresponds to the 3′-untranslated region (UTR) of the gene encoding a human SMN protein or corresponds to the 3′-UTR of the human SMN1 gene.

54. An isolated nucleic acid sequence comprising, in this order: an AAV 5′-ITR, a promoter, a gene encoding a SMN protein, a polyadenylation signal and an AAV 3′-ITR, wherein said isolated nucleic acid is configured to form a single-stranded AAV genome which is not self-complementary.

55. The isolated nucleic acid sequence according to claim 54 comprising, in this order: an AAV2 5′-ITR, the CAG promoter, a human SMN1 gene, a HBB2 polyadenylation signal and an AAV2 3′-ITR.

56. The isolated nucleic acid sequence according to claim 55, wherein said nucleic acid sequence comprises SEQ ID NO:1, SEQ ID NO:11, or a sequence that is at least 80% identical to SEQ ID NO:1 or SEQ ID NO:11.

57. A plasmid comprising the isolated nucleic acid sequence according to claim 54.

58. A method of treating spinal muscular atrophy (SMA) comprising administering the rAAV vector according to claim 46 to a subject in need of treatment, said rAAV vector comprising:
(i) a capsid selected from an AAV9 capsid and an AAVrh10 capsid; and
(ii) a single-stranded genome which is not self-complementary, said genome containing a nucleic acid sequence comprising, in this order:
an AAV
a CAG promoter;
a gene coding a human survival of motor neuron (SMN) protein;
optionally, a further regulatory element;
a HBB2 polyadenylation signal; and
an AAV 3′-ITR;
wherein the further regulatory element is not a WPRE.

59. The method according to claim 58, wherein the nucleic acid sequence comprises SEQ ID NO:1, SEQ ID NO:11 or a sequence that is at least 80% identical to SEQ ID NO:1 or SEQ ID NO:11

60. The method according to claim 58, wherein the rAAV vector is administered into the cerebrospinal fluid of a subject.

61. The method according to claim 58, wherein the rAAV vector is administered by intrathecal and/or intracerebroventricular injection.

62. The method according to claim 58, wherein said SMA is infantile SMA, intermediate SMA, juvenile SMA or adult-onset SMA.

63. A method of treating spinal muscular atrophy (SMA) comprising administering to a subject in need of treatment a rAAV vector according to claim 46, said rAAV vector comprising:
(i) an AAV9 capsid or an AAVrh10 capsid; and
(ii) a single-stranded genome which is not a self-complementary genome including a gene coding a human survival motor neuron (SMN) protein, wherein said genome does not comprise a WPRE and wherein said genome does not comprise a SV40 intron;
wherein the rAAV vector is administered into the cerebrospinal fluid of a subject.

64. The method according to claim 63, wherein the rAAV vector is administered by intrathecal and/or intracerebroventricular injection.

65. The method according to claim 63, wherein said SMA is infantile SMA, intermediate SMA, juvenile SMA or adult-onset SMA.


SEQUENCE LISTING (18 sequences)

SEQ ID NO: 1 - DNA, 3655 bp, artificial
  rAAV genome (7210)
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaactgggcggggagggccttcgtgcgtcgccgcgccgccgtccccttctccatctccagcctcggggctgccgcagggggacggctgccttcgggggggacggggcagggcggggttcggcttctggcgtgtgaccggcggcctctgctaaccatgttcatgccttcttctttttcctacagctcctgggcaacgtgctggttattgtgctgtctcatcattttggcaaagaattgccaccatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggtttcagacaaaatcaaaaagaaggaaggtgctcacattccttaaattaaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 2 - DNA, 145 bp, artificial
  AAV2 ITR-5′
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 3 - DNA, 378 bp, artificial
  CMV enhancer
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattacca

SEQ ID NO: 4 - DNA, 276 bp, artificial
  chicken beta actin promoter
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 5 - DNA, 960 bp, artificial
  chicken beta actin intron 1
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaactgggcggggagggccttcgtgcgtcgccgcgccgccgtccccttctccatctccagcctcggggctgccgcagggggacggctgccttcgggggggacggggcagggcggggttcggcttctggcgtgtgaccggcgg

SEQ ID NO: 6 - DNA, 94 bp, artificial
  rabbit beta globin intron2/exon2
cctctgctaaccatgttcatgccttcttctttttcctacagctcctgggcaacgtgctggttattgtgctgtctcatcattttggcaaagaatt

SEQ ID NO: 7 - DNA, 6 bp, artificial
  Kozak sequence
gccacc

SEQ ID NO: 8 - DNA, 885 bp, artificial
  hSMN1 ORF
atggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggtttcagacaaaatcaaaaagaaggaaggtgctcacattccttaaattaa

SEQ ID NO: 9 - DNA, 766 bp, artificial
  Human beta globin gene polyadenylation signal
attcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatc

SEQ ID NO: 10 - DNA, 145 bp, artificial
  AAV2 ITR-3′
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 11 - DNA, 4463 bp, artificial
  rAAV genome (7209)
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaactgggcggggagggccttcgtgcgtcgccgcgccgccgtccccttctccatctccagcctcggggctgccgcagggggacggctgccttcgggggggacggggcagggcggggttcggcttctggcgtgtgaccggcggcctctgctaaccatgttcatgccttcttctttttcctacagctcctgggcaacgtgctggttattgtgctgtctcatcattttggcaaagaattgccaccatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggtttcagacaaaatcaaaaagaaggaaggtgctcacattccttaaattaaggagtaagtctgccagcattatgaaagtgaatcttacttttgtaaaactttatggtttgtggaaaacaaatgtttttgaacatttaaaaagttcagatgttaaaaagttgaaaggttaatgtaaaacaatcaatattaaagaattttgatgccaaaactattagataaaaggttaatctacatccctactagaattctcatacttaactggttggttatgtggaagaaacatactttcacaataaagagctttaggatatgatgccattttatatcactagtaggcagaccagcagacttttttttattgtgatatgggataacctaggcatactgcactgtacactctgacatatgaagtgctctagtcaagtttaactggtgtccacagaggacatggtttaactggaattcgtcaagcctctggttctaatttctcatttgcaggaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactggggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 12 - DNA, 885 bp, artificial
  hSMN1co_ATUM
atggccatgagcagcggtggttcaggcggtggagtgcctgagcaagaggattcggtgctgttcaggaggggcaccggacagtccgacgactccgatatttgggatgataccgcactgattaaggcatacgacaaggccgtggcgtccttcaagcacgcgctgaagaatggcgacatctgcgaaacctcaggaaagcccaagactaccccgaagcgcaaaccggccaagaagaacaagtcgcagaagaagaacactgccgccagcctccaacagtggaaagtcggggacaagtgctccgccatctggtccgaggacggatgtatctacccggccaccattgcctccatcgacttcaagcgcgagacttgcgtggtcgtgtataccggatacggcaaccgcgaagaacagaatctcagcgatctgctgtcacccatctgcgaagtggcgaacaacatcgaacagaacgcccaggagaacgaaaacgagtcccaagtctccaccgacgaatccgagaactcgagatcacccgggaacaagtccgacaacattaagccgaagtctgccccctggaactccttccttccgcctccgccacctatgcccggacccagacttgggccggggaaacctggtctgaagttcaatggaccacctccgcctcctccacctcctcccccacacctcctgtcctgctggttgcccccgtttccctccggaccgcctattatcccaccaccgcctcctatctgcccggactccctggacgatgccgacgctctggggagcatgctgatctcgtggtacatgagcggataccacaccggctactacatgggattccggcagaaccagaaggaaggccggtgttcgcattcgctgaactga

SEQ ID NO: 13 - DNA, 885 bp, artificial
  hSMN1co_Genc3
atggcaatgagcagcggaggaagcggaggaggagttcctgaacaggaggacagcgtgctgttcaggagaggaaccggacagagcgacgacagtgacatctgggacgacaccgcactgatcaaagcctacgacaaggcagtggcaagctttaagcacgccctgaagaacggagatatttgtgagacaagcggcaagcccaaaaccacacccaaacgcaagcccgctaagaaaaacaagtcacagaagaagaacacagctgcctcactgcagcaatggaaggtgggagacaagtgcagcgcaatctggagcgaggacggatgtatctaccccgcaacaatagccagcatcgacttcaagagagaaacctgcgtggtggtgtacaccggctacggaaacagagaagagcagaacctgagcgacctgctgagccctatatgcgaggtggctaataacatcgagcaaaacgcccaggagaacgagaacgagagccaggttagcaccgatgagagcgaaaacagcagaagccccggcaacaaaagcgacaacatcaagcccaagagcgccccatggaacagcttcctgcctcctcctccacctatgcctggacctagactgggaccaggaaaacccggactgaaattcaacgggccaccccctccaccaccacctcctcctcctcatctgctgtcatgctggctccctcctttcccttccggacctcctatcatcccccctcctcctcctatctgccctgattctctcgacgacgccgacgctctgggatctatgctgatctcctggtacatgtccggctaccacaccggttactacatgggcttcagacagaaccaaaaggaaggccggtgttcccacagcctgaactga

SEQ ID NO: 14 - DNA, 885 bp, artificial
  hSMN1co_Genw4
atggccatgtcctccggaggaagcggaggaggcgtgcctgaacaggaggacagcgtgctgtttaggaggggcacaggccagagcgacgactccgacatctgggatgacaccgctctgatcaaggcctacgacaaggccgtggccagcttcaagcacgctctgaagaacggcgacatctgtgagacctccggcaagcccaagaccacacccaagaggaagcccgccaagaagaacaagtcccagaagaagaacaccgccgcttccctgcagcagtggaaggtgggcgacaagtgctccgctatctggtccgaggatggctgcatctaccccgccaccattgcctccatcgacttcaagagggagacctgcgtggtggtgtacaccggctacggcaacagggaggagcagaacctgagcgacctgctgagccctatctgcgaggtggctaacaacatcgagcagaacgcccaagagaatgagaacgagtcccaggtgagcacagacgagagcgagaattccaggtcccccggcaataagagcgacaacatcaagcccaagagcgccccctggaacagctttctgcctcctcccccccctatgcctggccctagactcggacccggaaaacccggcctgaagttcaacggacctccccctcctcctcctcctcctcctccccatctgctgagctgctggctgcccccttttccctccggacctcccatcattcctcctcctccccccatttgccccgactccctggacgatgccgacgctctgggctccatgctgatcagctggtacatgtccggctaccacaccggctactacatgggcttcaggcagaaccagaaagagggcaggtgctcccactccctgaactga

SEQ ID NO: 15 - DNA, 885 bp, artificial
  hSMN1co_MMV3.5
atggccatgagctctggagggtctggaggaggagtgcctgagcaggaggactctgtgctgttcagaagaggcacaggccagtctgatgattctgacatctgggatgacacagccctgatcaaggcctatgacaaggctgtggcttccttcaagcatgccctgaagaacggagacatctgtgagacttctggcaagccaaagaccacacccaagagaaagcctgccaagaagaacaagagccagaagaagaacactgctgccagcctgcagcagtggaaggtgggggacaagtgctctgctatctggtcagaggatggctgtatctaccctgccaccattgccagcattgacttcaagagagagacctgtgtggtggtgtacacaggctatggcaacagagaggagcagaacctgtctgacctgctgagccccatctgtgaggtggccaacaacattgagcagaatgcccaggagaatgagaatgagagccaggtgagcacagatgagtctgagaacagcagatctcctggcaacaagtctgacaatatcaagcccaagtctgccccctggaacagcttcctgccccctcctcctcctatgcctggccccagactgggacctggcaagcctggcctgaagttcaacggcccccctccccctccccctccccctccccctcacctgctgagctgctggctgccccccttcccctctggcccccccatcatcccccctcctccccctatctgccctgactctctggatgatgctgatgccctgggcagcatgctgatcagctggtatatgtctggctaccacacaggctactacatgggcttcagacagaaccagaaggagggcagatgcagccacagcctgaactga

SEQ ID NO: 16 - DNA, 574 bp, artificial
  HBB2 pA part
aaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatc

SEQ ID NO: 17 - DNA, 1000 bp, artificial
  hSMN1 3′-UTR
ggagtaagtctgccagcattatgaaagtgaatcttacttttgtaaaactttatggtttgtggaaaacaaatgtttttgaacatttaaaaagttcagatgttaaaaagttgaaaggttaatgtaaaacaatcaatattaaagaattttgatgccaaaactattagataaaaggttaatctacatccctactagaattctcatacttaactggttggttatgtggaagaaacatactttcacaataaagagctttaggatatgatgccattttatatcactagtaggcagaccagcagacttttttttattgtgatatgggataacctaggcatactgcactgtacactctgacatatgaagtgctctagtcaagtttaactggtgtccacagaggacatggtttaactggaattcgtcaagcctctggttctaatttctcatttgcaggaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactggggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaa

SEQ ID NO: 18 - DNA, 678 bp, artificial
  WPRE4
gagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctgga